(19) United States
(12) Patent Application Publication
(10) Pub. No.: US 2026/0317678 A1
(43) Pub. Date: Oct. 15, 2026

(54) PERFUSION BIOREACTOR TANGENTIAL FLOW FILTRATION

(71) Applicant: ASTRAZENECA AB, Södertälje (SE)

(72) Inventors: Jimmy VU, Gaithersburg, MD (US); Andrea SQUERI, Gaithersburg, MD (US); Kenneth LEE; Jon COFFMAN, Gaithersburg, MD (US)

(21) Appl. No.: 19/119,185

(22) PCT Filed: Oct. 9, 2023
(86) PCT No.: PCT/IB2023/060131

§ 371 Date: Apr. 8, 2025

Related U.S. Application Data
(60) Provisional application No. 63/378,970, filed on Oct. 10, 2022.

(51) Int. Cl. C12M 1/26; B01D 61/18; B01D 63/02; B01D 63/08; C12M 1/00

(52) U.S. Cl. CPC C12M 33/14; B01D 61/18; B01D 63/02; B01D 63/082; C12M 29/04; C12M 29/10; C12M 29/18; C12M 47/10; B01D 2311/2523; B01D 2311/2665; B01D 2313/243; B01D 2315/10

(57) ABSTRACT

Provided herein are systems and methods of enhancing recovery of a product using constant pressure and/or low flow rate tangential flow filtration.

### CROSS-REFERENCE TO RELATED APPLICATION

This application claims the priority benefit of U.S. Provisional Application No. 63/378,970, filed Oct. 10, 2022, which is incorporated herein by reference in its entirety.

### BACKGROUND OF THE DISCLOSURE

Tangential flow filtration (also referred to as cross-flow filtration or TFF) systems are widely used in the separation of particulates suspended in a liquid phase, and have important bioprocessing applications. Tangential flow systems are characterized by fluid feeds that flow across a surface of the filter, resulting in the separation of the feed into two components: a permeate component which has passed through the filter and a retentate component which has not. Compared to dead-end systems, TFF systems are less prone to fouling. Fouling of TFF systems may be reduced further by alternating the direction of the fluid feed across the filtration element, by backwashing the permeate through the filter, and/or by periodic washing.

Despite their advantages, TFF systems still suffer from drawbacks that lead to costly loss of product. Therefore, there exists a need to generate TFF systems that have reduced loss of products of interest.

### SUMMARY OF THE DISCLOSURE

The present disclosure provides a system for enhanced recovery of a product of interest during tangential flow filtration (TFF), the system comprising: (1) a feed reservoir; (2) a TFF unit comprising a pump in fluid contact with the fluid to be filtered, and at least one filter element for separating a liquid feed into permeate and retentate; and (3) a second pump which is in fluid contact with the permeate from the first filter element.

In one aspect, the system further comprises a second filter element in direct contact with the retentate and permeate from the first filter element. In another aspect, the filter elements are stacked in series. In another aspect, the one or more of the filter elements comprise a hollow fiber or cassette. In another aspect, the hollow fiber as a pore size of between 0.2-0.65 μM.

In another aspect, one or more of the pumps is a peristaltic, diaphragm, or magnetic levitation pump.

In another aspect, the system is configured to operate in a recirculation mode.

In another aspect, the feed reservoir is a perfusion bioreactor. In another aspect, the perfusion bioreactor is more than 1000 liters in size. In another aspect, the bioreactor is 5000 or 6000 liters in size. In another aspect, the system is configured for large-scale processing of products of interest.

The present disclosure also provides a method of minimizing retention of a product of interest in a permeate stream during TFF comprising passing a liquid feed containing the product of interest through a system described herein. In another aspect, the liquid feed comprises cells and a target product of interest. In another aspect, the product of interest is an antibody or antigen binding fragment thereof. In another aspect, the product of interest is recovered in the permeate. In another aspect, the liquid feed is passed through the system at a rate that minimizes cell shearing. In another aspect, the liquid feed is passed through the system at a rate of 1.8-8 mL/lumen/minute. In another aspect, the pressure difference between the retentate inlet and permeate is consistent with the system pressure following filtration.

The present disclosure also provides a method of minimizing retention of a product of interest in a permeate stream during TFF comprising passing a liquid feed through a system described herein at a flow rate at least one-third slower than a conventional TFF flow rate. In another aspect, the flow rate provide a shear rate of about 1800 s⁻¹.

In another aspect, the method further comprises air sparging the system. In another aspect, the air comprises about 10-80% dissolved oxygen. In another aspect, the liquid feed is sparged with air prior to contact with the filter element. In another aspect, the sparging occurs at a rate necessary to maintain greater than 10% dissolved oxygen throughout the TFF system. In some aspects, the sparging comprises introducing oxygen with a bubble diameter of from about 1 μm to about 10 μm. In some aspects, the sparging comprises introducing oxygen with a bubble diameter of about 1 μm or about 10 μm. In another aspect, the liquid feed comprises cells and a target product of interest. In another aspect, the sparging minimizes lactate production by the cells. In another aspect, the product of interest is an antibody or antigen binding fragment thereof. In another aspect, the method further comprises recovering the product of interest in the permeate. In some aspects, the sparging increases specific productivity of the cells as compared to cells without sparging. In some aspects, the sparging increases specific productivity of the cells to greater than about 0.0034 gmL⁻¹day⁻¹ as compared to cells without sparging. In some aspects, the sparging increases specific productivity of the cells from at least 0.0034 gmL⁻¹day⁻¹ to about 0.0044 gmL⁻¹day⁻¹. In another aspect, the pressure difference between the retentate inlet and permeate is consistent with the system pressure following filtration.

### BRIEF DESCRIPTION OF THE DRAWINGS

FIG. 1 shows a schematic of a typical TFF system.

FIG. 2 shows product retention and corresponding yield loss during small-scale TFF. FIG. 2A shows product titer in the permeate and retentate. FIG. 2B shows the percent yield of product in TFF.

FIG. 3 shows a schematic of the high performance (HPTFF) system of the present disclosure. The TFF system employs a second pump in connection with the permeate. The TFF system can employ one or more filter cassettes.

FIG. 4 shows increased yield of AZ-1 (FIG. 4A) and AZ-2 (FIG. 4B) using HPTFF and lowflow TFF compared to traditional TFF.

FIG. 5 shows culture performance of AZ-1 for different viable cell density target of 90 and 120 million cells per mL (FIG. 5A), viability (FIG. 5B), glucose levels (FIG. 5C), pH (FIG. 5D), osmolality (FIG. 5E), and lactate levels (FIG. 5F) using HPTFF.

FIG. 6 shows dissolved oxygen levels under typical TFF conditions (FIG. 6A) and with air sparging (FIG. 6B), and a schematic (FIG. 6C).

FIG. 7 shows culture performance of AZ-3 for typical TFF with and without air sparging for viable cell density (FIG. 7A), viability (FIG. 7B), lactate levels (FIG. 7C), total product titer (FIG. 7D), specific productivity (FIG. 7E), and product retention (FIG. 7F.

### DETAILED DESCRIPTION

The present disclosure provides a highly effective approach to minimize retention of products of interest during TFF. In some aspects, the disclosure provides methods of minimizing product loss caused by a pressure drop during large-scale processing of perfusion cell cultures. In another aspect, the cultures are larger than 1000 L cultures. In some aspects, the disclosure provides methods employing two or more pumps in the TFF system to minimize pressure drop. In some aspects, the disclosure provides methods employing low flow rates and air sparging to reduce retention of the product of interest.

### I. Definitions

In order that the present disclosure can be more readily understood, certain terms are first defined. As used in this specification, except as otherwise expressly provided herein, each of the following terms shall have the meaning set forth below. Additional definitions are set forth throughout the specification.

It is to be noted that the term “a” or “an” refers to one or more of that entity; for example, “a feed medium,” is understood to represent one or more feed mediums. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

The term “bioreactor,” as used herein, refers to any suitable vessel or other means of producing and maintaining a biological cell culture including, but not limited to, a perfusion/perfused bioreactor. The bioreactors of the present disclosure are used for large-scale production of products of interest. In some aspects, the bioreactor volume is greater than 1000 L. In some aspects, the bioreactor volume is 1000 L, 1500 L, 2000 L, 2500 L, 3000 L, 3500 L 4000 L, 4500 L, 5000 L, 5500 L, 6000 L, 6500 L, 7000 L, 7500 L, 8000 L, 8500 L, 9000 L, 9500 L, or 10,000 L.

The terms “perfusion” as used herein, refer to a fermentation or cell culture process used to produce a targeted biological product, e.g., an antibody or recombinant protein, in which a high concentration of cells within a sterile chamber receive fresh growth medium continually as the spent medium which may contain a targeted biological product that is harvested.

The term “cut-off size” or “molecular weight cut-off” as used herein with respect to ultrafiltration membranes refers to the molecular weight of a molecule or particle of which 90% is retained by the membrane.

The expression “spiral-wound filter element” refers to a filtration membrane that is spirally wound about a core. A spiral-wound filter element may be contained within a housing and may alternately be referred to as a spiral-wound filter module.

“Pressure drop” refers to the drop in pressure (e.g., psid) from the retentate inlet and the permeate.

“Flux” is the area-normalized flow rate.

“Permeate flux” is the area normalized flow rate of permeate in a permeate channel (e.g., Liters/hr/m², 1 mh).

“Cross-flow flux” is the area normalized average flow rate of retentate in a feed channel (e.g., Liters/min/m², LMM).

“Cross-flow” is the retentate flow rate between inlet and outlet of the feed channel in a filter or a series of filters. Unless otherwise stated, “cross-flow” refers to an average cross-flow.

The term “shear” refers to a strain in the structure of a substance that is produced by pressure.

The term “shear rate” refers to the rate at which a progressive shearing deformation is applied (e.g., s−1).

The terms “feed,” “feed sample” and “feed stream” refer to the solution being introduced into a filtration module for separation.

The term “separation” generally refers to the act of separating the feed sample into two streams, a permeate stream and a retentate stream.

The terms “permeate” and “permeate stream” refer to that portion of the feed that has permeated through the membrane.

The terms “retentate” and “retentate stream” refer to the portion of the solution that has been retained by the membrane, and the retentate is the stream enriched in a retained species.

“Feed channel” refers to a conduit in a filtration assembly, module or element for a feed.

“Permeate channel” refers to a conduit in a filtration assembly, module, or element for a permeate.

The expression “flow path” refers to a channel comprising a filtration membrane (e.g., ultrafiltration membrane, microfiltration membrane) through which the solution being filtered passes (e.g., in a tangential flow mode). The flow path can have any topology which supports tangential flow (e.g., straight, coiled, arranged in zigzag fashion). A flow path can be open, as in an example of channels formed by hollow fiber membranes, or have one or more flow obstructions, as in the case, for example, of rectangular channels formed by flat-sheet membranes spaced apart by woven or non-woven spacers.

“TFF assembly,” “TFF system” and “TFF apparatus” are used interchangeably herein to refer to a tangential flow filtration system that is configured for operation in a single-pass mode and/or a recirculation mode (e.g., full or partial recirculation) and/or alternating flow mode.

“Single leaf” spirals are spiral-wound filter elements that can be formed with one continuous feed channel. They are generally made with one sheet of membrane.

“Multi-leaf” spirals are spiral-wound filter elements that have multiple feed channels. They are generally made with more than one sheet of membrane; but can be made with 1 membrane sheet also.

A “cassette holder” refers to a compression assembly for one or more cassettes. Typically, when a cassette holder contains more than one cassette, the cassettes are configured for parallel processing, although, in some embodiments, the cassettes can be configured for serial processing.

A “cassette” refers to a cartridge or flat plate module comprising filtration (e.g., ultrafiltration or microfiltration) membrane sheet(s) suitable for TFF processes.

“Filtration membrane” refers to a selectively permeable membrane capable of use in a filtration system, such as a TFF system.

The term “microfiltration membranes” and “MF membranes” are used herein to refer to membranes that have pore sizes in the range between about 0.1 micrometers to about 10 micrometers.

“Fluidly connected” refers to a plurality of spiral-wound membrane TFF modules that are connected to one another by one or more conduits for a liquid, such as, a feed channel, retentate channel and/or permeate channel.

“Product” refers to a target compound. In some aspects, a product will be a biomolecule (e.g., protein) of interest, such as a monoclonal antibody (mAb).

“Processing” refers to the act of filtering (e.g., by TFF) a feed containing a product of interest and subsequently recovering the product (e.g., in a purified form). The product can be recovered from the filtration system (e.g., a TFF assembly) in either the retentate stream or permeate stream depending on the product's size and the pore size of the filtration membrane.

The expressions “parallel processing”, “processing in parallel”, “parallel operation” and “operation in parallel” refer to processing a product in a TFF assembly that contains a plurality of processing units that are fluidly connected by distributing the feed directly from a feed channel or manifold to each of the processing units in the assembly.

The expressions “serial processing”, “processing in series”, “serial operation” and “operation in series” refer to processing a product in a TFF assembly that contains a plurality of processing units that are fluidly connected by distributing the feed directly from the feed channel to only the first processing unit in the assembly. In serial processing, each of the other, subsequent processing units in the assembly receives its feed from the retentate line of the preceding processing unit (e.g., the retentate from a first processing unit serves as the feed for a second, adjacent processing unit).

As used herein “perfusion” or “perfusion culture” or “perfusion culture process” refers to continuous flow of a physiological nutrient solution at a steady rate, through or over a population of cells. As perfusion systems generally involve the retention of the cells within the culture unit, perfusion cultures characteristically have relatively high cell densities, but the culture conditions are difficult to maintain and control. In addition, since the cells are grown to and then retained within the culture unit at high densities, the growth rate typically continuously decreases over time, leading to the late exponential or even stationary phase of cell growth. This continuous culture strategy generally comprises culturing mammalian cells, e.g., non-anchorage dependent cells, expressing a polypeptide and/or virus of interest during a production phase in a continuous cell culture system. In some aspects, the perfusion culture is a large-scale culture. In some aspects, the culture is greater than a 1000 liter culture. In another aspect, the culture is a 3,000, 4,000, 5,000, 6,000, 7,000, 8,000, 9,000, or 10,000 liter culture.

Various aspects of the disclosure are described in further detail in the following subsections.

### II. High Performance Tangential Flow Filtration (HPTFF)

Perfusion systems and methods, in contrast with fed-batch systems, involve continuous filtration of cell culture media. During filtration, products of interest (e.g., target proteins, such as monoclonal antibodies), and optionally other soluble components, such as cellular waste products (e.g., lactic acid and ammonia), are removed from the cell culture media. Perfusion systems present unique challenges over fed-batch systems as the cells contained in a perfusion system pass repeatedly through filtration equipment, which can cause physical damage to the cells and which, in turn, can reduce productivity of the system. It is desirable to minimize cell damage during filtration in perfusion systems so as to retain as many cells as possible for ongoing production of the target protein.

Tangential flow filtration (TFF) is a separation process that uses membranes to separate components in a liquid solution or suspension on the basis of size, molecular weight or other differences. TFF is used in perfusion processes to remove target products of interest, e.g., proteins, from cell culture media, while retaining cells within the media. In TFF processes, fluid is pumped tangentially along the membrane surface and particles, molecules, or cells that are too large to pass through the membrane are rejected and returned to a process tank. TFF processes can involve additional passes of the fluid across the membrane (e.g., recirculation) until the process fluid is sufficiently clarified, concentrated or purified. The cross-flow nature of TFF minimizes membrane fouling, thus permitting high volume processing per batch. The membranes are contained within filter elements that can be of a variety of configurations, such as spiral-wound filter elements and cassette filter elements.

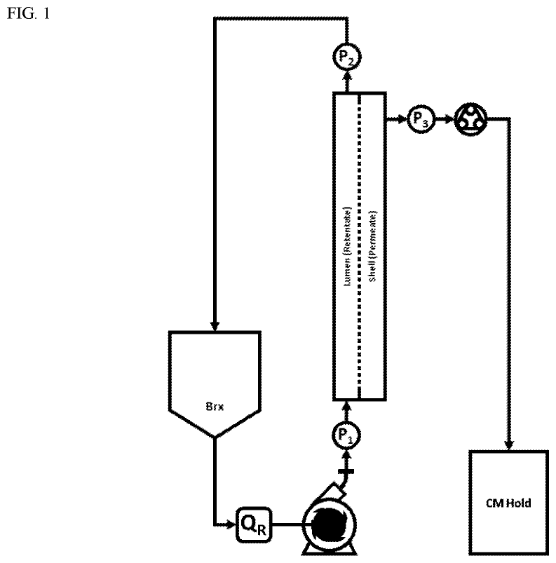

A typical TFF system is shown in FIG. 1. Pressurized feed from a feed tank is connected to the feed port of the spiral-wound filter module or manifold of the cassette filter. Feed flows through the membrane lined feed channel of the TFF device(s) under control of a pump. Some of the solvent from the feed stream flows through the face of the membrane into the permeate channel and carries with it a portion of the permeable species (e.g., product of interest and waste product). The remaining concentrated feed stream flows out of the module or manifold through the retentate port. The permeate flowing from the module's permeate port is directed to a location that is dependent on the process, where it is either collected (e.g., as with product of interest) or discarded (e.g., as with waste product).

However, under large-scale production conditions, e.g., greater that bioreactor volumes of 1000 L, TFF systems experience a pressure drop between the retentate inlet and the permeate which causes the product of interest to remain in the retentate due to the large processing volume. This pressure drop leads to product of interest being retained in the retentate, which in turn leads to loss of product recovery.

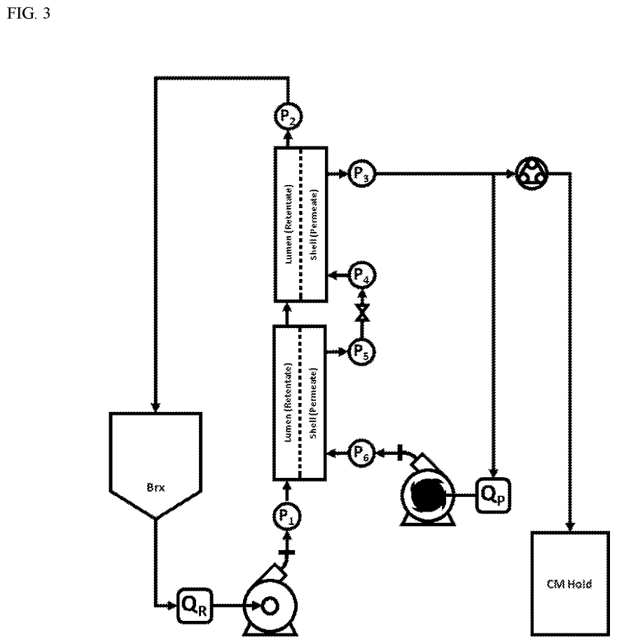

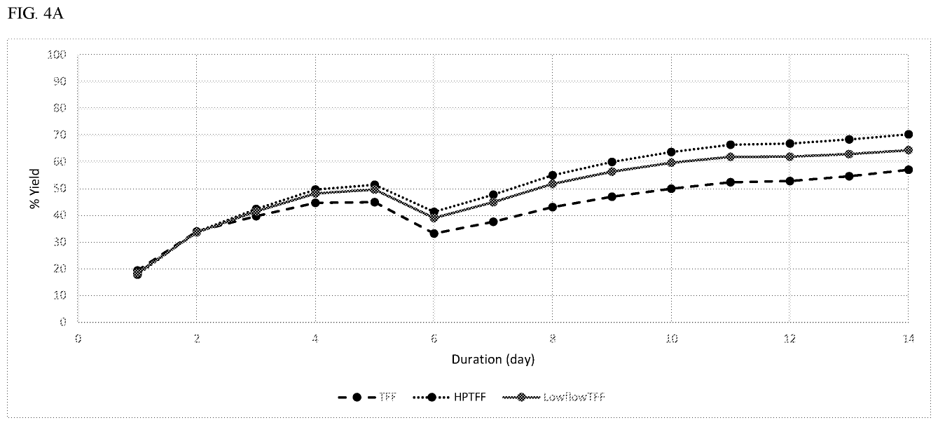

To overcome the pressure drop, the high performance TFF (HPTFF) systems described herein include two or more pumps for recirculating retentate through all or part of the system and at least one conduit for recirculating (e.g., carrying) retentate (FIG. 3). In one aspect, one pump is located at the retentate inlet and one pump is in contact with the permeate. A flow meter can be used to provide a process value for the pump or valve to control the amount of retentate that is recirculated. Alternatively, or in addition, a valve or pump and/or flow meter can be positioned on the permeate outlet or in the flow line carrying permeate out of the system to control or limit permeate flow.

Maximum achievable flux during TFF system operation can be obtained by selection of an adequate transmembrane pressure (TMP) for permeate discharge. This applies to pressure-dependent and mass-transfer-limited regions of operation. For spiral-wound filters, attainment of the desired TMP is determined by measurement at the end of the module. For cassettes with, for example, two permeate outlets, attainment of the desired TMP is determined by the average feed channel pressure. The transmembrane pressure must be sufficient to support both the pressure drop through the membrane and the maximum pressure to discharge permeate from the permeate channel. Alternatively, or in addition, maximum achievable flux during a TFF system operation can be obtained by selection of an adequate permeate flow rate for permeate discharge. The permeate flow rate can be controlled to a constant value by use of a permeate valve or pump.

Current TFF devices used in perfusion systems include hollow fiber devices and open-channel cassette devices, also referred to as plate-and-frame devices. Examples of currently-available filtration devices for perfusion systems include, but are not limited to, XCell™ ATF System (Repligen, Waltham, Mass.) and KrosFlo® Perfusion System (Spectrum Laboratories, Rancho Dominguez, Calif.), which are hollow fiber devices, and Prostak™ Microfiltration Modules (MilliporeSigma, Billerica, Mass.), which are cassette devices. These devices contain open feed channels, so as to limit physical damage to cells in the feed stream, and both devices require high cross-flow rates to minimize fouling (i.e., the accumulation of particles along the wall of membrane). Membrane fouling and pressure drops in the TFF system reduce product recovery because the passage of target proteins and waste materials through the membrane (i.e., sieving) is reduced. In some aspects, the hollow fibers have a pore size of 0.2, 0.25, 0.3, 0.35, 0.4, 0.45, 0.5, 0.55, 0.6, 0.65, 0.7, 0.75, 0.8, 0.85, 0.9, 0.95, or 1.0 μM.

In a perfusion process, cell culture media is introduced to a feed-side of the membrane. As the liquid feed (e.g., the cell culture media) travels across the surface of the membrane, it is separated into permeate and retentate. Specifically, target products of interest pass through the membrane and are recovered from the permeate exiting the filter through a collection tube. Cells are retained and are recovered from the retentate exiting the filter. The cell culture media in the retentate can then be returned to a bioreactor, and the product of interest contained in the permeate can be collected in a separate vessel for further processing.

The perfusion system can include a TFF system having one or more than one spiral-wound filter element or cassette filter element described herein. In systems having more than one filter element, the filter elements can be fluidly connected in series or in parallel, or both.

TFF systems can be operated in a recirculation mode, where all or a portion of the retentate is returned to the filter element(s) for further filtration. In a perfusion system, following filtration, the retentate can be returned to a bioreactor where the cell culture media may be maintained for some period of time before being recirculated through the TFF system.

The feed pumps as illustrated in FIG. 3 can be configured to operate in a recirculation mode. The feed pump can be a pump that is not damaging to cells, such as a magnetic levitation pump, a diaphragm pump, a peristaltic pump, or a rotary vane pump. Examples of suitable magnetic levitation pumps include, but are not limited to, Levitronix® Puralev® Series pump (Levitronix Technologies, Framingham, Mass.). Examples of suitable diaphragm pumps include Repligen XCell™ ATF pump (Repligen, Waltham, Mass.). Examples of suitable peristaltic pumps include Watson Marlow Series 500 and Series 600 pumps (Watson Marlow, Wilmington, Mass.).

In one aspect, the disclosure relates to a method of passing a liquid feed through the HPTFF system described herein containing at least one filter element, separating the liquid feed into permeate and retentate in the filter element; and recovering the permeate and at least a portion of the retentate from the filter element. The liquid feed can comprise a cell culture media, containing cells and a target product of interest. The target product of interest can be recovered in the permeate and the cells can be retained in the retentate.

The process can include recirculating at least a portion of the retentate through the filter element. Recirculation can be performed on an ongoing basis or at regular intervals to continually harvest product from the cell culture media.

The retentate that is being recirculated can be returned to any upstream location in or before the HPTFF system (e.g., a bioreactor located upstream of the HPTFF system). In one aspect, the retentate is recirculated to the feed tank. In another aspect, the retentate is recirculated to the feed line near the feed pump before the feed inlet on the HPTFF system.

In some aspects, the methods described herein comprise performing perfusion under low flow rates to overcome a pressure drop in the TFF system. Low flow rates correlate with increased product quality due to reduced shear rates. In one aspect, the low flow rate is about one-third the typical rate of TFF systems. In another aspect, the traditional flow rate correlates to a shear rate of about 1800 s⁻¹. Therefore, in another aspect the low flow rate correlates to a shear rate of about 600 s⁻¹.

In some aspects, low flow TFF causes oxygen deprivation in the HPTFF system due to the length of time the feed stream spends outside of the bioreactor. Therefore, in some aspects, the feed stream is sparged with air. In some aspects, the feed stream is sparged with between 10 and 80% dissolved oxygen. In some aspects, the feed stream is sparged with 10%, 20%, 30%, 40%, 50%, 60%, 70% or 80% dissolved oxygen. In some aspects, the oxygen is introduced with a bubble diameter of about 1 μm. In some aspects, the oxygen is introduced with a bubble diameter of about 10 μm. In some aspects, the oxygen is introduced with a bubble diameter of from about 1 μm to about 10 μm.

In some aspects, the disclosure provides for the purification of any product of interest. In some aspects, the product of interest is a protein. Thus, in some aspects, the disclosure relates to a perfusion process for harvesting target proteins from a liquid feed containing host cells. The target proteins can be monoclonal antibodies, which are separated from the host cells by TFF and recovered from the permeate of the filter element(s).

### EXAMPLES

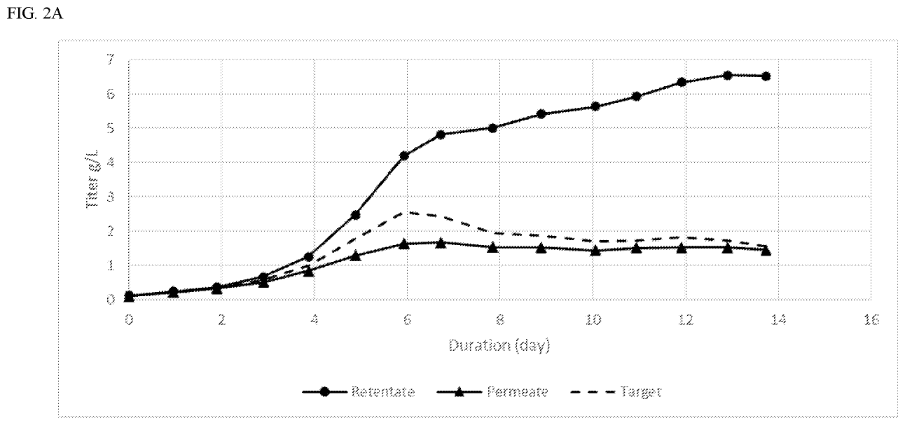

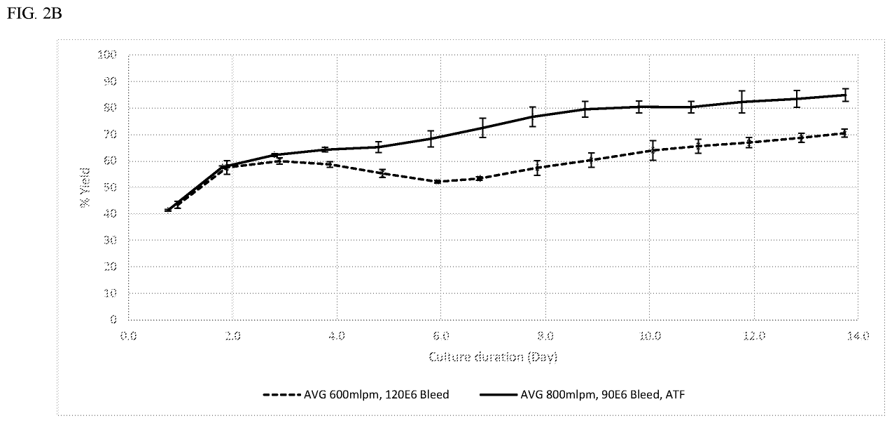

A bioreactor comprising cells producing AZ-1, a monoclonal antibody, was subjected to standard TFF as shown in FIG. 1. The bioreactor volume was 2 liters and the TFF was run at flow rate corresponding to 1360 s⁻¹, using a Repligen S04-P20U-10-N hollow fiber filter. As shown in FIG. 2, using standard TFF conditions, as culture duration and the concomitant amount of protein produced increased, the titer in the TFF retentate increased with respect to the target titer (FIG. 2A). This increase over target titer caused a significant decrease in the yield of the recovered protein (FIG. 2B).

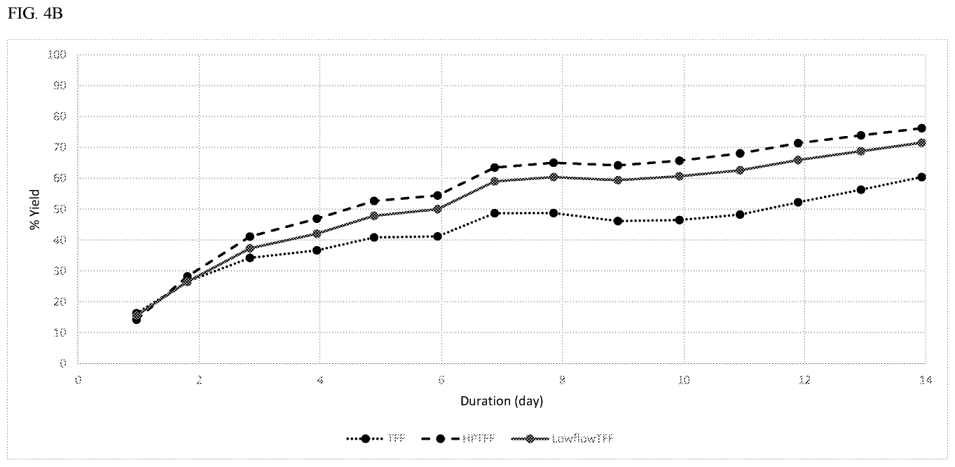

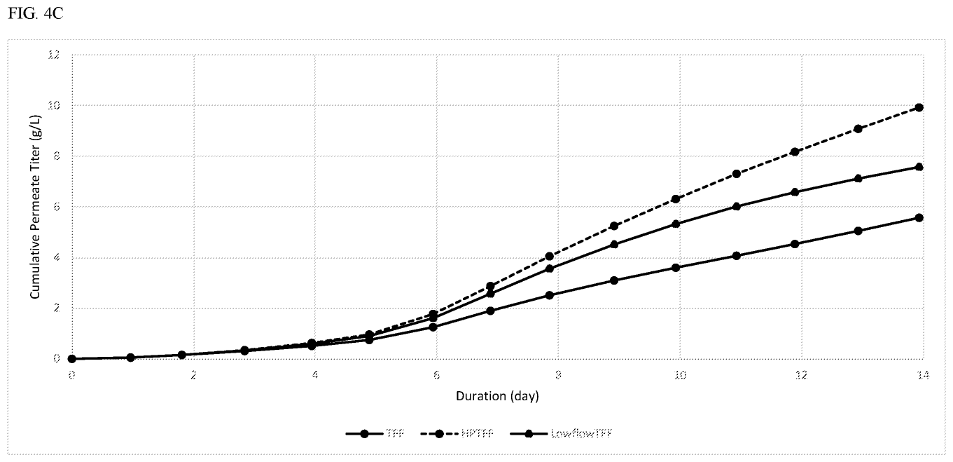

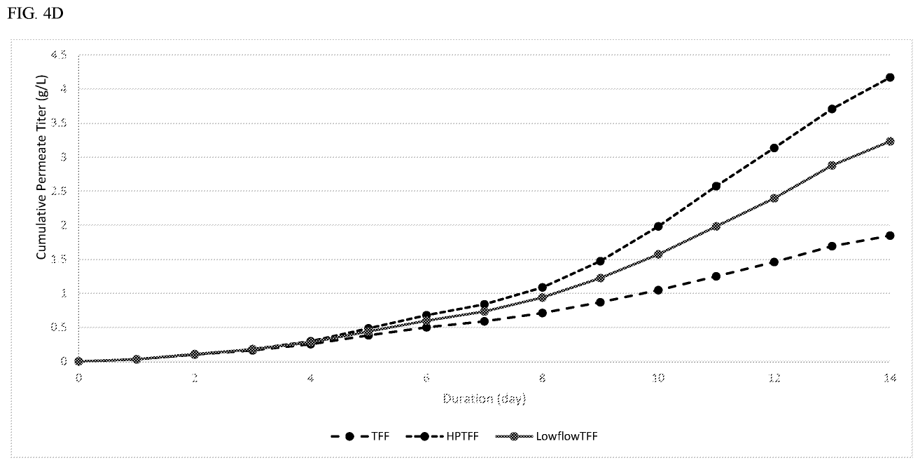

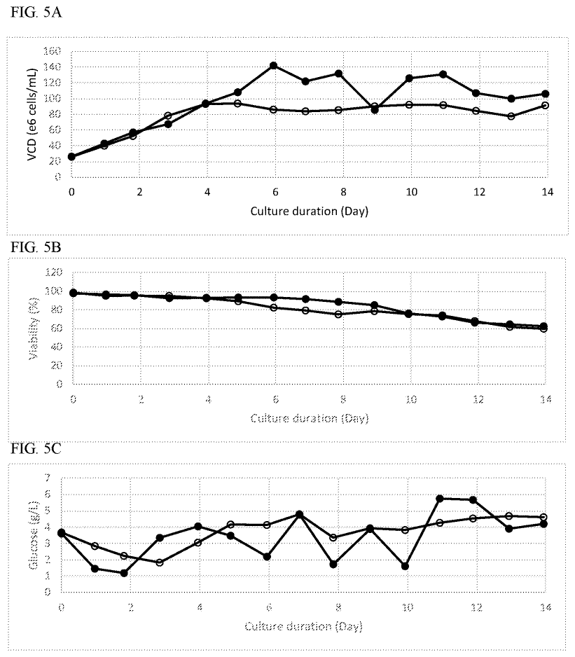

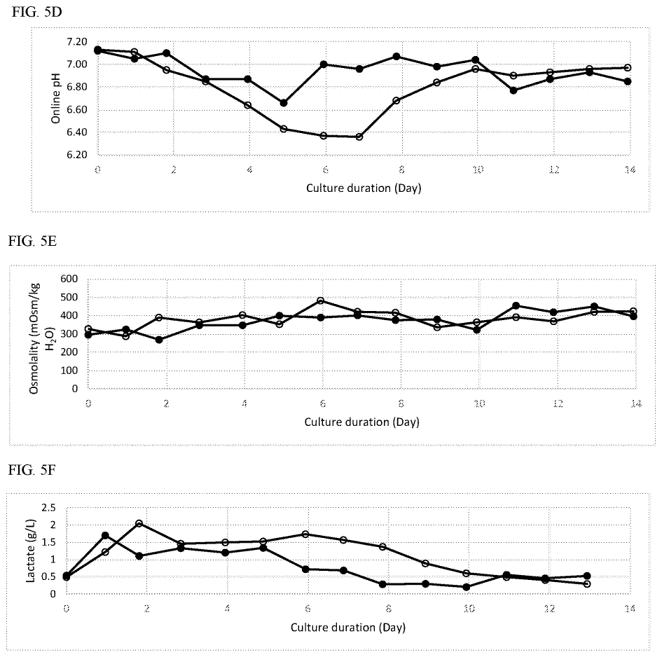

AZ-1 and AZ-2 (a monoclonal antibody) were then subjected to HPTFF and lowflow TFF as described herein. A bioreactor feed from a 2L reactor was run at flow rate corresponding to 1360 s⁻¹, using a Repligen S04-P20U-10-N hollow fiber filter. For the lowflowTFF, the flow rate correspond to a shear rate of 600 s⁻¹. As shown in FIG. 4, when clarified using the HPTFF and lowflow systems described herein, the percent yield of AZ-1 (FIG. 4A) and AZ-2 (a monoclonal antibody, FIG. 4B) was significantly increased compared to standard TFF. Culture performance was unaffected for AZ-1 as measured by viable cell density, viability, glucose levels, pH, osmolality, and lactate levels (FIG. 5A-5F).

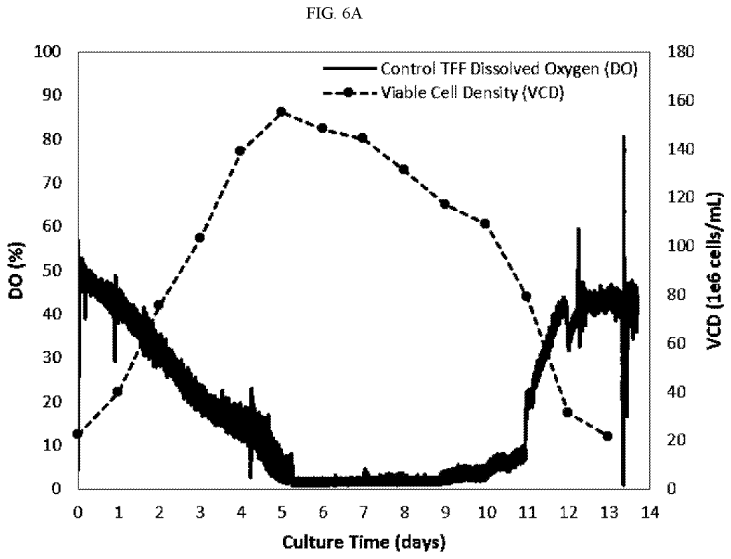

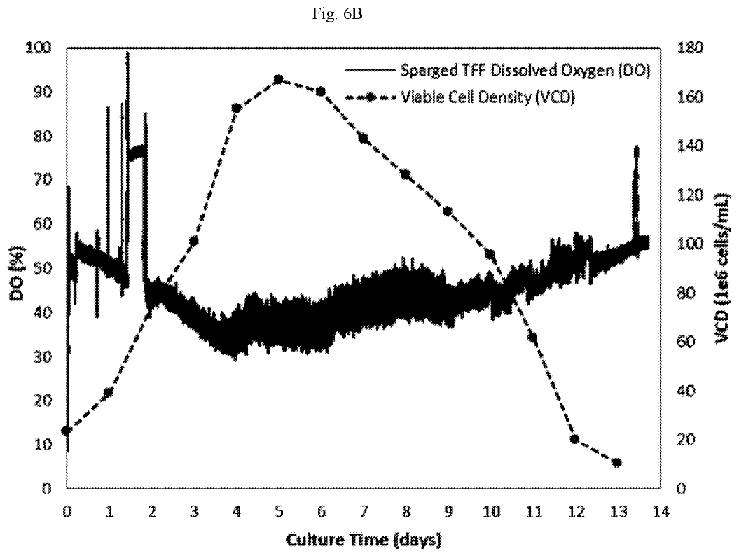

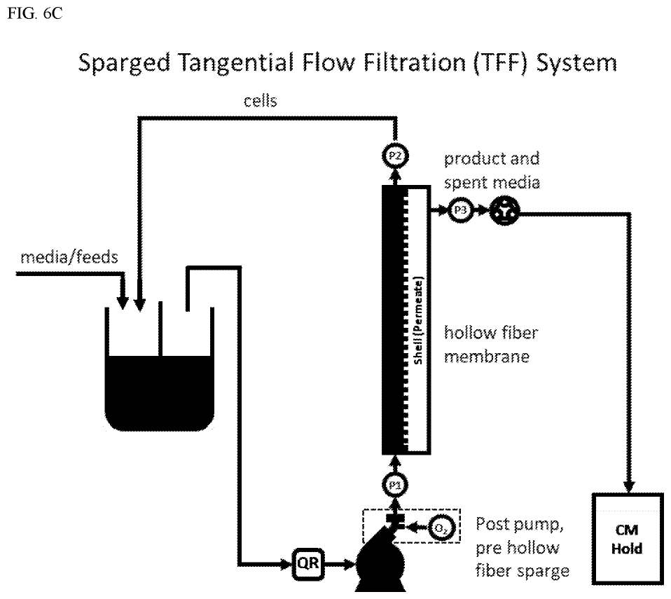

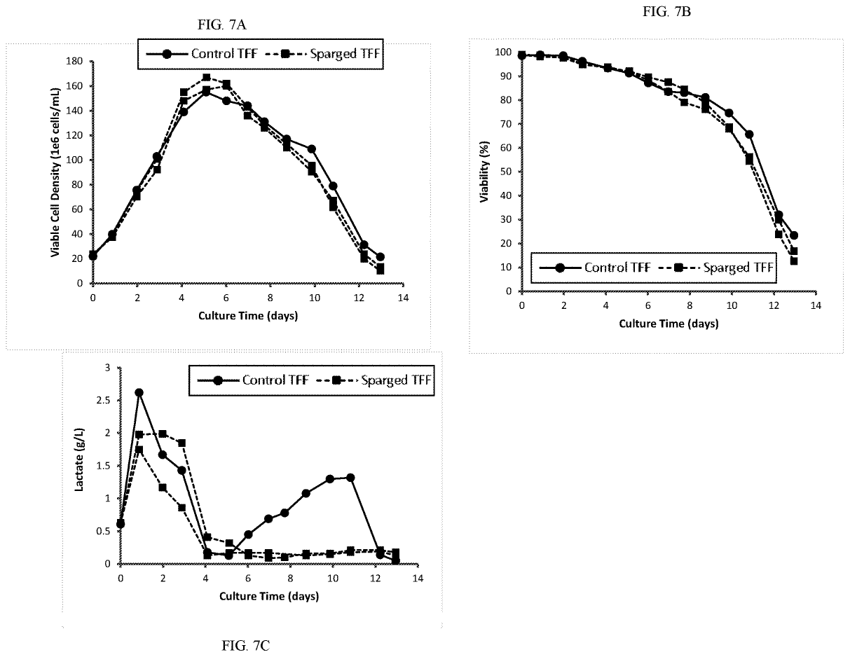

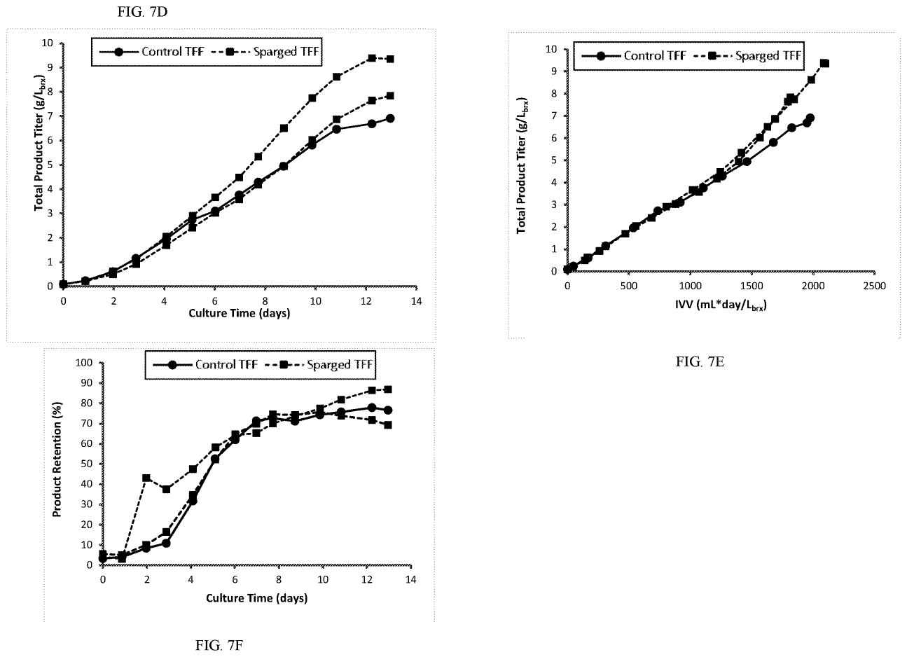

Low feed stream flow rate was also analyzed to overcome retention of product in the retentate. Low flow rates of one-third the typical rate TFF were analyzed. As shown in FIG. 6A, dissolved oxygen levels were depleted during typical TFF, but normal levels were reinstated upon sparging the recirculation loop with air containing 21% dissolved oxygen. Low dissolved oxygen levels decreased specific productivity (FIG. 7). Culture performance of typical TFF on AZ-3, a bispecific antibody, showed that Air sparging overcame the negative effects of oxygen deprivation on specific productivity without affecting viable cell density and cell viability. (FIG. 7A-7D). Further, the specific productivity per cell volume was approximately 0.0044gmL⁻¹day⁻¹ in sparged TFF compared to 0.0034 gmL⁻¹day⁻¹ in control TFF.

What is claimed is:

1. A system for enhanced recovery of a product of interest during tangential flow filtration (TFF), the system comprising: (1) a feed reservoir; (2) a TFF unit comprising a pump in fluid contact with the fluid to be filtered, and at least one filter element for separating a liquid feed into permeate and retentate; and (3) a second pump which is in fluid contact with the permeate from the first filter element.

2. The system of claim 1, further comprising a second filter element in direct contact with the retentate and permeate from the first filter element.

3. The system of claims 1 or 2, wherein one or more of the filter elements comprise a hollow fiber or cassette.

4. The system of claim 3, wherein the filter elements are stacked in series.

5. The system of any one of claims 1-4, wherein the hollow fiber as a pore size of 0.2-0.65 μM.

6. The system of any one of claims 1-5, wherein one or more of the pumps is a peristaltic, diaphragm, or magnetic levitation pump.

7. The system of any one of claims 1-6, wherein the system is configured to operate in a recirculation mode.

8. The system of any one of claims 1-7, wherein the feed reservoir is a perfusion bioreactor.

9. The system of any one of claims 1-8, wherein the perfusion bioreactor is more than 1000 liters in size.

10. The system of claim 9, wherein the bioreactor has a volume of 5000 or 6000 liters.

11. The system of any one of claims 1-10, wherein system is configured for large-scale processing of products of interest.

12. A method of minimizing retention of a product of interest in a permeate stream during TFF comprising passing a liquid feed containing the product of interest through the system of any one of claims 1-11.

13. The method of claim 12, wherein the liquid feed comprises cells and a target product of interest.

14. The method of claim 12 or 13, wherein the product of interest is an antibody or antigen binding fragment thereof.

15. The method of any one of claims 12-14, further comprising recovering the product of interest in the permeate.

16. The method of any one of claims 12-15, wherein the liquid feed is passed through the system at a rate that minimizes cell shearing.

17. The method of claim 16, wherein the flow rate is 1.8-8 mL/lumen/min.

18. The method of any one of claims 12-17, wherein the pressure difference between the retentate inlet and permeate is consistent with the system pressure following filtration.

19. A method of minimizing retention of a product of interest in a permeate stream during TFF comprising passing a liquid feed through the system of any one of claims 1-11 at a flow rate at least one-third slower than a conventional TFF flow rate.

20. The method of claim 19, wherein the flow rate corresponds to a shear rate of about 1800 s⁻¹.

21. The method of claim 19 or 20, further comprising air sparging the system.

22. The method of claim 21, wherein the air comprises about 10-80% dissolved oxygen.

23. The method of any one of claims 19-22, wherein the liquid feed is sparged with air prior to contact with the filter element.

24. The method any one of claims 21-23, wherein sparging occurs at a rate necessary to maintain above 10% dissolved oxygen throughout the TFF system.

25. The method of any one of claims 21-24, wherein the sparging comprises introducing oxygen with a bubble diameter of from about 1 μm to about 10 μm.

26. The method of any one of claims 21-25, wherein the sparging comprises introducing oxygen with a bubble diameter of about 1 μm or about 10 μm.

27. The method of any one of claims 19-26, wherein the liquid feed comprises cells and a target product of interest.

28. The method of any one of claims 21-25, wherein the sparging minimizes lactate production by the cells.

29. The method of any one of claims 19-26, wherein the product of interest is an antibody or antigen binding fragment thereof.

30. The method of any one of claims 21-29, wherein the sparging increases specific productivity of the cells as compared to cells without sparging.

31. The method of any one of claims 21-30, wherein the sparging increases specific productivity of the cells to greater than about 0.0034 gmL⁻¹day⁻¹ as compared to cells without sparging.

32. The method of any one of claims 21-31, wherein the sparging increases specific productivity of the cells from at least 0.0034 gmL⁻¹day⁻¹ to about 0.0044 gmL⁻¹day⁻¹.

33. The method of any one of claims 19-32, further comprising recovering the product of interest in the permeate.

34. The method of any one of claims 19-33, wherein the system pressure prior to the filter element(s) is consistent with the system pressure after filtration.
